(19) United States
(12) Patent Application Publication
(10) Pub. No.: US 2007/0066638 A1
(43) Pub. Date: Mar. 22, 2007

(54) RYANODINE RECEPTOR BLOCKERS FOR TREATING PAIN

(76) Inventors: Cun-Jian Dong, Irvine, CA (US); William A. Hare, Tustin, CA (US); John E. Donello, Dana Point, CA (US)

Correspondence Address: ALLERGAN, INC., 2525 DUPONT DRIVE, T2-7H, IRVINE, CA, 92612-1599, US

(21) Appl. No.: 11/530,537

(22) Filed: Sep. 11, 2006

Related U.S. Application Data
(60) Provisional application No. 60/717,803, filed on Sep. 16, 2005.

(51) Int. Cl. A61K 31/485; A61K 31/4166; A61K 31/13

(52) U.S. Cl. 514/282; 514/389; 514/662

(57) ABSTRACT

The present invention provides a method of preventing or ameliorating pain in a mammal comprising administering to said mammal suffering from or at risk of suffering a noxious action causing said pain, an effective amount of a ryanodine antagonist, such as dantrolene, to inhibit or prevent pain.

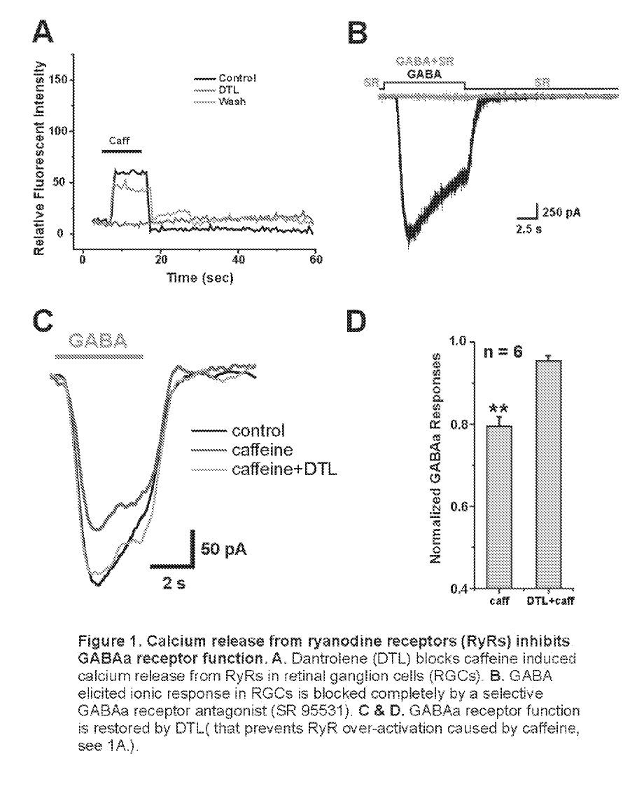

### CROSS REFERENCE TO RELATED APPLICATIONS

This application is based on, and claims the benefit of, U.S. Provisional Application No. 60/717,803, filed Sep. 16, 2005, and which is incorporated herein by reference.

### BACKGROUND OF THE INVENTION

1. Field of the Invention

This invention relates to the use of ryanodine receptor blockers or antagonists for treating pain. In particular, dantrolene, is used in a method for treating pain according to the present invention.

2. Description of the Related Art

Inhibitory mechanisms, particularly those mediated by GABAergic pathways, are essential in suppressing the development of allodynia and hyperalgesia under normal conditions. There is evidence that loss of inhibition can lead to the development of pain. For example, intrathecal administration of GABAa antagonists has been shown in animal models to induce tactile allodynia and hyperalgesia.

Overactivation of NMDA receptors has also been shown to play a significant role in allodynia and hyperalgesia. Under normal conditions, GABAergic inhibition is essential to prevent over-activation of NMDA receptors.

Non-steroidal anti-inflammatory drugs (NSAIDS) and opiates are the main classes of drugs that are utilized in pain relief. However both possess undesirable side effects. NSAIDS are know to cause gastrointestinal irritation and opiates are known to be addictive.

Aromatic compounds which antagonize the pain-enhancing effects of E-type prostaglandins are disclosed in U.S. Pat. Nos. 5,811,459; 5,834,458 and 5,843,942. However, the need for compounds which relieve pain, without side effects, continues to exist.

### BRIEF SUMMARY OF THE INVENTION

A new class of compounds, that are structurally different than NSAIDS and opiates, and useful in the relief of pain, have now been found.

Dantrolene, a skeletal muscle relaxant, has been found to be an antagonist of the ryanodine receptor-channel complex (See Biochemistry 2001, 40, 531-542). Dantrolene blocks calcium release from ryanodine channels when it binds to the receptor.

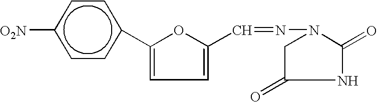

Dantrolene is 1-[[5-(p-Nitrophenyl)furfurylidene]amino]hydantoin.

The compounds utilized in the method of the present invention are useful in the treatment of pain such as the pain associated with joint conditions (such as rheumatoid arthritis and osteoarthritis), post-operative pain, post-partum pain, the pain associated with dental conditions (such as dental caries and gingivitis), the pain associated with burns (including sunburn), the treatment of bone disorders (such as osteoporosis, hypercalcaemia of malignancy and Paget's disease), the pain associated with sports injuries and sprains (such as back pain) and other painful conditions.

The present invention provides a method of preventing abnormal calcium release from ryanodine receptors present in CNS neurons. Abnormal calcium release from ryanodine receptors suppresses the GABAa receptor function. Blocking this release with an antagonist of the ryanodine receptor, e.g. dantrolene, restores GABAa receptor function and thus prevents and/or ameliorates pain. Thus, the present invention utilizes a ryanodine receptor antagonist to prevent or ameliorate painful reactions caused by noxious provocations that induce excessive calcium release from intracellular stores via ryanodine receptor channels. The method comprises administering to the mammal either systemically, topically, epidurally or by intrabulbar injection an effective amount of one or more ryanodine receptor antagonists, such as dantrolene.

In the method of the present invention, the active compounds (or mixtures or salts thereof) are administered in accordance with the present invention to the patient admixed with an acceptable carrier. Any suitable, e.g., conventional, acceptable carrier may be employed. A carrier is acceptable if it has substantially no long term or permanent detrimental effect on the patient to which it is administered. Examples of acceptable carriers include physiological saline and other aqueous media. In accordance with the invention, the active compounds are preferably soluble in the carrier which is employed for their administration, so that the active compounds are administered to the patient in the form of a solution. Alternatively, a suspension of the active compound or compounds (or salts thereof) in a suitable carrier may also be employed.

In accordance with the invention the active compounds (or mixtures or salts thereof) are administered in an acceptable carrier in sufficient concentration so as to deliver an effective amount of the active compound or compounds to the patient. Preferably, the therapeutic solutions contain one or more of the active compounds in a concentration range of approximately 0.0001% to approximately 10% (weight by volume) and more preferably approximately 0.005% to approximately 0.5% (weight by volume).

Any method of administering drugs directly to a patient may be employed to administer, in accordance with the present invention, the active compound or compounds to the patient to be treated. The primary effect on the mammal resulting from the direct administering of the active compound or compounds to the patient is preferably a reduction in pain. More preferably, the active useful compound or compounds are injected into the patient.

An exemplary formulation is shown below in Table 1. The abbreviation q.s. means a quantity sufficient to effect the result or to make volume.

TABLE I

| Ingredient | Amount (% W/V) |
|---|---|
| Active Compound in accordance | about 0.0001 to about 1 |
| with the invention | |
| Preservative | 0-0.10 |
| Vehicle | 0-40 |
| Tonicity Adjustor | 1-10 |
| Buffer | 0.01-10 |
| PH Adjustor | q.s pH 4.5-7.5 |
| Antioxidant | as needed |
| Purified Water | as needed to make 100% |

Various preservatives may be used in the preparation described in Table I above. Preferred preservatives include, but are not limited to, benzalkonium chloride, chlorobutanol, thimerosal, phenylmercuric acetate, and phenylmercuric nitrate.

Likewise, various preferred vehicles may be used in such preparation. These vehicles include, but are not limited to, polyvinyl alcohol, povidone, hydroxypropyl methyl cellulose, poloxamers, carboxymethyl cellulose, hydroxyethyl cellulose, and purified water.

Various buffers and means for adjusting pH may be used so long as the resulting preparation is pharmaceutically acceptable. Accordingly, buffers include but are not limited to, acetate buffers, citrate buffers, phosphate buffers, and borate buffers. Acids or bases may be used to adjust the pH in these formulations as needed.

In a similar vein, pharmaceutically acceptable antioxidants include, but are not limited to, sodium metabisulfite, sodium thiosulfate, acetylcysteine, butylated hydroxyanisole, and butylated hydroxytoluene.

The solution may be administered to the patient as often as necessary to obtain the desired concentration that affords prevention and/or amelioriation of pain. For chronic treatments, the drug would be administered as frequently as necessary to maintain desired concentration or range of concentrations at all times. In other words, the solution (or other formulation) which contains the ryanodine antagonist as the active ingredient, is administered to the patient as often as necessary to maintain the beneficial effect of the active ingredient in the patient. Those skilled in the art will recognize that the frequency of administration depends on the precise nature of the active ingredient and its concentration in the formulation. Within these guidelines it is contemplated that the formulation of the present invention will be administered to the patient approximately once or twice daily.

This new method is particularly effective when administered as a prophylactic treatment, i.e. before the pain has ocurred, or before long-term progression of the disease state, which causes pain, has taken place. Without wishing to be held to a particular theory regarding the role that the compounds of the present invention play in preventing pain, applicants hypothesize that the compounds and methods described inhibit the intracellular Ca+2 release. (See for example U.S. Pat. No. 5,891,911.)

Thus it is further contemplated that the compounds of the present invention can advantageously be used in combination with compounds that prevent pain associated with abnormal calcium release. Such compounds include NMDA antagonists especially memantine.

### BRIEF DESCRIPTION OF THE DRAWING FIGURE

The Figure shows the effect of dantrolene on the caffeine-induced release of calcium via ryanodine receptors in retinal ganglion cells.

### DETAILED DESCRIPTION OF THE INVENTION

The present invention relates to a method of using ryanodine receptor antagonists to prevent and/or ameliorate acute or chronic clinical pain in an individual which pain results from injuries caused by acute and chronic pathological provocations.

Two major types of clinical pain are inflammatory pain and neuropathic pain. Inflammatory pain is caused by tissue damage or inflammation and neuropathic pain by nervous system lesions that result from neural injury, diabetes, alcoholism and cancer therapy.

According to a further feature of the invention there is provided a method for the relief of both inflammatory and neuropathic pain in the animal (including a human) body in need of such treatment which comprises administering to said body an effective amount of a ryanodine blocker or antagonist or a pharmaceutically-acceptable salt thereof.

As mentioned above, the ryanodine blocker or antagonist is useful in treating the pain which, for example, accompanies inflammatory conditions such as rheumatoid arthritis and osteoarthritis. It will generally be administered so that a daily dose in the range, for example, 0.1 mg to 75 mg per kg body weight is received, given, if required, in divided doses. In general lower doses will be administered when a parenteral route is employed. Thus, for example, for intravenous administration, a dose in the range, for example, 0.05 mg to 30 mg per kg body weight will generally be used. Similarly, for administration by inhalation, a dose in the range, for example, 0.05 mg to 25 mg per kg body weight will be used.

By virtue of their ability to relieve pain, the ryanodine blockers or antagonists are of value in the treatment of certain inflammatory and non-inflammatory disease which are currently treated with a cyclooxygenase-inhibitory non-steroidal anti-inflammatory drug (NSAID) such as indomethacin, ketorolac, acetylsalicyclic acid, ibuprofen, sulindac, tolmetin and piroxicam. Co-administration of a ryano dine blocker or antagonist with a NSAID can result in a reduction of the quantity of the latter agent needed to produce a therapeutic effect. Thereby the likelihood of adverse side-effects from the NSAID such as gastrointestinal effects are reduced.

The compounds of the invention may also be used with other anti-inflammatory agents such as an inhibitor of the enzyme 5-lipoxygenase (such as those described in European Patent Applications Nos. 0351194, 0375368, 0375404, 0375452, 037547, 0381375, 0385662, 0385663, 0385679, 0385680.)

The ryanodine blocker or antagonist may be administered in pharmaceutically-acceptable compositions which, in addition, include one or more other therapeutic or prophylactic agents known to be of value for the treatment of pain. Thus, for example, a known opiate pain-killer (such as dextropropoxyphene, dehydrocodeine or codeine) or an antagonist of other pain or inflammation mediators, such as bradykinin, takykinin and calcitonin gene related peptides (CGRP), or an alpha₂adrenoceptor agonist, a GABA_{b} receptor agonist, a calcium channel blocker, a sodium channel blocker, a CCK_{b} receptor antagonist, a neurokinin antagonist or an antagonist and modulator of the action of glutamate at the NMDA receptor may usefully also be present in a pharmaceutical composition of the invention.

As mentioned above, excessive release of calcium from intracellular stores under disease conditions is believed suppress the GABAa receptor function, which is expected to cause the NMDA receptor overactivation. Both suppression of GABAa receptors and overactivation of NMDA receptors have been associated with pain induction.

The ryanodine receptor blocker or antagonist may be administered locally, e.g. by intrathecal or epidural administration. Many of the agents of the invention can be administered systemically, e.g., orally, or intravenously, or by intramuscular injection.

The composition used in these therapies may also be in a variety of forms. These include, for example, solid, semi-solid, and liquid dosage forms, such as tablets, pills, powders, preserved or non-preserved liquid solution or suspension, liposomes, suppositories, injectable and infusible solutions. The compositions also preferably include conventional pharmaceutically acceptable carriers which are known to those of skill in the art.

The following non-limiting examples describe assays and measurements used in 1) evaluating efficacy of the ryanodine blockers and/or antagonists utilized in the method of this invention for preventing or ameliorating pain, 2) selecting ryanodine antagonists other than dantrolene for use in the method of this invention and 3) demonstrating that abnormal calcium release from ryanodine receptors suppresses GABAa receptor function.

### EXAMPLE 1

### Alleviation of Chronic Pain

A model for chronic pain (in particular peripheral neuropathy such as causalgia) involves the surgical ligation of the L5 (and optionally the L6) spinal nerves on one side in experimental animals. Rats recovering from the surgery gain weight and display a level of general activity similar to that of normal rats. However, these rats develop abnormalities of the foot, wherein the hindpaw is moderately everted and the toes are held together. More importantly, the hindpaw on the side affected by the surgery appears to become sensitive to pain from low-threshold mechanical stimuli, such as that producing a faint sensation of touch in a human, within about 1 week following surgery. This sensitivity to normally non-painful touch is called “tactile allodynia” and lasts for at least two months. The response includes lifting the affected hindpaw to escape from the stimulus, licking the paw and holding it in the air for many seconds. None of these responses is normally seen in the control group.

Rats are anesthetized before surgery. The surgical site is shaved and prepared either with betadine or Novacaine. Incision is made from the thoracic vertebra Xlll down toward the sacrum. Muscle tissue is separated from the spinal vertebra (left side) at the L4-S2 levels. The L6 vertebra is located and the transverse process is carefully removed with a small rongeur to expose the L4-L6 spinal nerves. The L5 and L6 spinal nerves are isolated and tightly ligated with 6-0 silk thread. The same procedure is done on the right side as a control, except no ligation of the spinal nerves is performed.

A complete hemostasis is confirmed, then the wounds are sutured. A small amount of antibiotic ointment is applied to the incised area, and the rat is transferred to the recovery plastic cage under a regulated heat-temperature lamp. On the day of the experiment, at least seven days after the surgery, six rats per test group are administered dantrolene by intraperitoneal (i.p.) injection or oral gavage. For i.p. injection, the compounds are formulated in approximately 50% DMSO and given in a volume of 1 ml/kg body weight is tested at doses ranging between 30 and 3000 ng/kg. For oral gavage, a volume equal to 1 ml/kg body weight of an appropriate concentration (ie. 1 mg/ml for a 1 mg/kg dose) formulated in approximately 50% DMSO was injected using an 18-gauge, 3-inch gavage needle that is slowly inserted through the esophagus into the stomach.

Tactile allodynia is measured prior to and 30 minutes after drug administration using von Frey hairs that are a series of fine hairs with incremental differences in stiffness. Rats are placed in a plastic cage with a wire mesh bottom and allowed to acclimate for approximately 30 minutes. The von Frey hairs are applied perpendicularly through the mesh to the mid-plantar region of the rats' hindpaw with sufficient force to cause slight buckling and held for 6-8 seconds. The applied force has been calculated to range from 0.41 to 15.1 grams. If the paw is sharply withdrawn, it is considered a positive response. A normal animal will not respond to stimuli in this range, but a surgically ligated paw will be withdrawn in response to a 1-2 gram hair. The 50% paw withdrawal threshold is determined using the method of Dixon, W. J., Ann. Rev. Pharmacol. Toxicol. 20:441-462 (1980). The post-drug threshold is compared to the pre-drug threshold and the percent reversal of tactile sensitivity is calculated based on a normal threshold of 15.1 grams. Dantrolene is able to reduce the response to the tactile stimuli that indicate tactile allodynia. Compared to a saline solution, dantrolene reverses the allodynic pain at an i.p. dose of 100 ng/kg, at 300 ng/kg, at 100 mg/kg and at 3000 ng/kg.

### Example 1(a)

### Sulprostone, NMDA and Phenylephrine Models

In mice, it is possible to induce transient allodynia by treating mice with chemical sensitizers. An interperitoneal injection (of a 1 ml/kg volume) of phenylephrine (an alphal receptor agonist) or sulprostone (a EP1 and EP3 prostaglandin receptor agonist) will induce transient allodynia. Alternatively, an intrathecal injection of NMDA (in a 5 ul volume) will also induce transient allodynia. The injected mice are assessed for allodynia once every 5 minutes over a 15-50 min period post injection by light stroking of the flank with a paintbrush. The allodynia response was ranked as follows: 0. No response; 1, mild squeaking with attempts to move away from the paintbrush; 2, vigorous squeaking, biting at the paintbrush and strong efforts to escape. Data is expressed as the average total score for each group. (Each animal can have a maximum score of 16 over the 50-min period.) * indicates significant difference (P<0.05) relative to sulprostone, NMDA or phenylephrine-treated rats.

Sulprostone model

| Treatment | Dose | Mean pain score |
|---|---|---|
| Vehicle | | 5.8 ± 0.4* |
| Sulprostone (I.P) | 300 ng/kg | 14.0 ± 0.5 |
| Sulprostone + Dantrolene | 300 ng/kg/3 mg/kg | 12.6 ± 1.5 |
| Sulprostone + Dantrolene | 300 ng/kg/10 mg/kg | 9.5 ± 1.9* |
| Sulprostone + Dantrolene | 300 ng/kg/20 mg/kg | 4.8 ± 0.9* |

Phenylephrine model

| | | |
|---|---|---|
| Treatment | Dose | Mean pain score |

| | | |
|---|---|---|
| Vehicle | | 4.0 ± 1.4* |
| PE | 100 ng/kg | 11.3 ± 0.9 |
| PE + Dantrolene | 100 ng/kg/3 mg/kg | 9.4 ± 1.6 |
| PE + Dantrolene | 100 ng/kg/10 mg/kg | 6.5 ± 1.2* |

NMDA model

| Treatment | Dose | Mean pain score |
|---|---|---|
| Vehicle | | 5.8 ± 0.4* |
| NMDA | 100 ng | 14.2 ± 1.1 |
| NMDA + Dantrolene | 100 ng/3 mg/kg | 9.3 ± 0.8* |
| NMDA + Dantrolene | 100 ng/10 mg/kg | 7.0 ± 1.0* |

### Example 1(b)

### Capsaicin Model

Intraplantar injection of capsaicin, the active ingredient in red hot chili peppers, will induce neurogenic inflammation and transient allodynia.

Methods/subjects: Eight to ten male Sprague-Dawley rats (Charle Rivers, Sprague-Dawley, 200-300 grams) were used in these experiments. Rats received a unitlateral intradermal injection (10 ul) of capsaicin (0.1%) superficially in the mid-plantar surface of the hind paw. A successful injection was confirmed by the apperance of a bleb after intraplantar capsaicin administartion. Capsaicin, the pungent ingredient in hot peppers, has been used to produce hyperalgesia (mildly painful sensory stimuli are perceived as highly painful) in humans. When applied topically or injected into the skin, capsaicin produces burning pain, hyperalgesia to heat within the immediate vicinity of capsaicin application, and mechanical hyperalgesia within a large sorrounding (Hohmann AG, et al.). On the rat hind paw, there are no major subjective behaviour or clinical observations such as marked redness, limping, chronic licking and swelling associated with this model except for the immediate bleb formation of the given agent on the plantar surface right after the injection and as reported in the literature, short term tactile allodynia, extreme thermal hypoalgesia which persist for one week. On the day of the experiment, dantrolene (3 mg/kg) or vehicle was administered IP at 1 ml/kg 30 minutes before capsaicin administration, and then tested for allodynia at 15, 30 and 60 minute time points thereafter.

Allodynia Assessment: Rats were placed in Plexiglas cages positioned over an elevated wire mesh platform and habituated to the testing environment for 15 to 25 min before testing. Tactile allodynia was assessed using a single von frey filament (5.18) with a bending force of 10 grams (Dixon). Tactile allodynia refers to a nocifensive behavior elicited by a light touch or innocuous (here, mechanical) stimulus and was operationally defined as a lowering of the threshold for paw withdrawal from punctate mechanical stimulation (Hohmann AG, et al.). The filaments were positioned in contact with the hind paw for a duration of 5 s or until a withdrawal response occurred and repeated 10 consecutive times. This procedure was repeated three times to obtain a total of thirty responses and then averaged. Contralateral testing was also done; no capsaicin was given. Data are presented as paw withdrawl frequency (%). * indicates significant difference (P<0.05) relative to vehicle-treated rats.

| Injected | | | Uninjected | | Dantrolene |
|---|---|---|---|---|---|
| paw | Vehicle | Dantrolene | paw | Vehicle | (3 mg/kg IP) |
| Baseline | 4 ± 1.1 | 7 ± 0.9 | Baseline | 4 ± 1.5 | 5 ± 1 |
| before | | | | | |
| Caps. | | | | | |
| 15 min | 37 ± 1.5 | 36 ± 3.7 | 15 min. | 6 ± 1 | 5 ± 1.1 |
| 30 min | 38 ± 2.7 | 8 ± 1.7 * | 30 min. | 2 ± 1.6 | 2 ± 1.1 |
| 60 min | 10 ± 1.2 | 7 ± 2.1 | 60 min. | 2 ± 1.1 | 3 ± 1.3 |

### Example 1(c)

### Rotarod

The Rotamex® 4/8 Rota-rod is a device to test the motor performance decrement, performance time, partial or complete ataxia, and the motor coordination in laboratory animals. The principle of this system is to measure the time each animal manages to maintain walking on the moving rod. Drugs or conditions that cause muscular relaxation will interfere with this coordination and agility, causing the animal to fall from a safe height to a grid below.

The animals were conditioned before performing the test. Animals were required to pass three stages of conditioning on the Rota-rod prior to inclusion in the experiment. During the first stage of conditioning, animals were required to perch on the stationary rod for 30 seconds to accustom themselves to the environment.

Rats that fail to perch for 30 seconds will fall to the grid below. The fall alone typically serves as an adequate aversive stimulus and the animals learn to stay on the perch.

In the second stage of conditioning, the animals that passed the first stage of conditioning were then allowed to walk at a constant speed of 5-rpm for 90 seconds. Any animal that fell off the rod during this trial were allowed to fall to the grid below. Animals are required to stay on the rod for 90 seconds in three consecutive trials out of six total trials, or else they were excluded from the study.

In the final stage of conditioning, about 10-30 minutes after the last trial for each animal that passed the first two stages of conditioning, the animals were placed back on the rod in groups of four and allowed to run in an acceleration mode with a start speed of 5 rpm and an end speed of 15 rpm for 90 seconds. Each animal was given up to six trials to stay on greater than 80 seconds, three consecutive times. Most animals achieve this within 4-5 trials. A mean performance time was calculated for all animals that managed to stay on the rotating rod for at least 80 seconds. Typically, less than 10% of animals that begin conditioning are excluded due to poor performance in the conditioning procedure. Conditioned animals were then returned to their cages overnight and three baselines in acceleration mode (5-15 RPM) were taken the following morning (Day 0). Mean performance times were calculated and used as pre-drug baseline values. Following the acquisition of pre-drug baseline values, conditioned animals were administered vehicle or dantrolene, IP. Fifteen minutes post-dosing, the animals were placed back on the rod and the mean performance time in acceleration mode (5-15 RPM) is measured again. Data are presented as mean time on the rotating bar. * indicates significant difference (P<0.05) relative to vehicle-treated rats.

| Treatment | Dose | Mean time on bar |
|---|---|---|
| Vehicle | | 89.67 ± 0.33 |
| Dantrolene | 3 mg/kg | 74.33 ± 7.47 |
| Dantrolene | 10 mg/kg | 79.67 ± 5.43 |
| Dantrolene | 20 mg/kg | 37.67 ± 10.4* |

### Example 1(d)

### Activity Monitor

Locomotor activity of male Sprague-Dawley rats (Charles River, Wilmington, Mass.) weighing approximately 300 grams was measured thirty minutes following intraperitoneal injection of vehicle or test compound. Test animals were placed in a dark chamber and a Digicom analyzer (Omnitech Electronic, Columbus, Ohio), which records the number of interruptions of an array of 32 photoelectric beams in the X and Y orientation, quantitated exploratory behavior during a five-minute period. Horizonal activity (HD) and total distance (TD) are presented, as well as % vehicle values. * indicates significant difference (P<0.05) relative to vehicle-treated rats.

| | | | % | | % |
|---|---|---|---|---|---|
| Treatment | Dose | HA | vehicle | TD | vehicle |
| Vehicle | | 1366.8 ± 191.8 | | 488.2 ± 89.3 | |
| Dantrolene | 10 | 1125.6 ± 114.5 | 82.4% | 290 ± 48.4 | 59.4% |
| | mg/kg | | | | |

### EXAMPLE 2

### Assay for Selecting Ryanodine Antagonists other than Dantrolene

Assays for determining ryanodine antagonist may be conducted following procedures modified from that described by Laver et al., (J. Physiol. 537:763-778, 2001). Briefly, purified ryanodine receptor-channel complexes are incorporated into planar phospholipid bilayers with resting calcium gradient similar to that in a normal neuron at rest (100 nM cytoplasmic and 1 mM luminal). The level of channel activation can be determined in the presence of various ligands that activate ryanodine receptors. Effective antagonistic action of the compounds to be selected can be determined by a reduction of agonist-induced activation of the channel. The specificity of the antagonists can be determined by commercially available standard screens, such as NovaScreens.

### EXAMPLE 3

As reported in the Figure, this example shows that abnormal calcium release from ryanodine receptors (RyRs) in CNS neurons suppresses significantly GABAa receptor function. Blocking this release with a RyR antagonist dantrolene restores GABAa receptor function. These results suggest novel mechanisms that can lead to allodynia and hyperalgesia: homogenic (NMDA receptor mediated) and heterogenic (mediated by non-NMDA receptors, such as MGluR1 or 5) pathways both of which can suppress GABAa receptor function by stimulating calcium release from RyRs. Suppression of the GABAa receptor is expected not only to change significantly excitation-inhibition balance in general but also to cause specifically NMDA receptor overactivation by producing a positive feedback loop between NMDA receptors and GABAa receptors.

This example demonstrates that RyR is an excellent drug target for the treatment of pain. RyR modulators that inhibit or attenuate calcium overrelease from RyRs, such as dantrolene, can be used for the treatment of these indications.

The foregoing description details specific methods and compositions that can be employed to practice the present invention, and represents the best mode contemplated. However, it is apparent for one of ordinary skill in the art that further compounds with the desired pharmacological properties other than dantrolene may be utilized in the method of the present invention. For example, ryanodine antagonist such as procaine, ruthenium red, tetracaine, benzocaine, imperatoxin inhibitor, protamine sulfate, iberiotoxin, 8N-cADPR, DHBP, 2-aminoethyl-methanesulfonate, etc. may be substituted in the method of this invention. Additionally, the above ryanodine antagonists can be prepared according to methods known in the art or in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

What is claimed is:

1. A method of preventing or ameliorating pain in acute and chronic disorders in human patients comprising administering to said patients suffering from said disorders an effective amount of a compound that is a ryanodine receptor antagonist in pharmaceutically acceptable vehicle to prevent or ameliorate pain.

2. The method of claim 1 wherein said disorder is a result of overactivation of glutamate receptors.

3. The method of claim 2 wherein said glutamate receptor is the NMDA receptor.

4. The method of claim 3 wherein said NMDA receptor is in the pain pathway.

5. The method of claim 1 wherein said disorder is caused by suppression of the GABAa receptor function.

6. The method of claim 5 wherein said GABAa receptor is in the pain pathway.

7. The method of claim 1 wherein said pain is inflammatory pain caused by tissue damage or inflammation.

8. The method of claim 1 wherein said pain is neuropathic pain resulting from neural injury, diabetes, alcoholism or cancer therapy.

9. The method of claim 1 wherein said compound is administered in combination with a NMDA antagonist, a GABAa receptor agonist or enhancer, or a Na⁺ channel blocker.

10. The method of claim 9 wherein said NMDA antagonist is memantine.

11. The method of claim 1 wherein said compound is dantrolene.

12. The method of claim 1 wherein said compound is administered in an amount sufficient to achieve a serum concentration of from 0.01 nM to 5 μM.

13. The method of claim 1 wherein said compound is administered orally, intrathecally, or by intramuscular injection.

14. The method of claim 1 wherein said compound is in an aqueous solution, suspension, ointment, gel or jelly.

15. The method of claim 13 wherein said compound is administered intrathecally in combination with morphine, clonidine or baclofen.

16. The method of claim 8 wherein said pain results from diabetic neuropathy and said compound is administered at a dose that lacks muscle relaxant or sedative effect.
